(19) United States
(12) Patent Application Publication
(10) Pub. No.: US 2014/0316746 A1
(43) Pub. Date: Oct. 23, 2014

(54) REAL-TIME SIGNAL PROCESSING SYSTEM AND METHOD BASED ON MULTI-CHANNEL INDEPENDENT COMPONENT ANALYSIS

(71) Applicant: National Chiao Tung University, Hsinchu (TW)

(72) Inventors: Wai-Chi Fang, Hsinchu (TW); Wei-Yeh Shih, Hsinchu (TW); Jui-Chien Liao, Hsinchu (TW); Kuan-Ju Huang, Hsinchu (TW); Chiu-Kuo Chen, Hsinchu (TW)

(73) Assignee: National Chiao Tung University, Hsinchu (TW)

(21) Appl. No.: 14/257,199

(22) Filed: Apr. 21, 2014

(30) Foreign Application Priority Data
Apr. 22, 2013 (TW) 102114145

(51) Int. Cl. A61B 5/00; A61B 5/0476; A61B 5/04

(52) U.S. Cl. CPC A61B 5/7203; A61B 5/04012; A61B 5/0476
USPC 702/191

(57) ABSTRACT

A real-time signal processing system and method based on multi-channel independent component analysis (ICA). A one-pass recursive ICA processor uses a computation module to perform multi-channel ICA on a set of first data to generate a plurality of second data and third data. A noise removing module uses the computation module to identify noise in the second data and remove the identified noise to generate a plurality of fourth data. A reconstruction module uses the computation module to reconstruct the set of first data based on the fourth data and the third data to generate a plurality of fifth data. The one-pass recursive ICA processor, the noise removing module, the reconstruction module and the computation module are all implemented on a single chip, such that the one-pass recursive ICA processor, the noise removing module and the reconstruction module share the same computation module to save hardware resources.

### FIELD OF THE INVENTION

The present invention relates to a real-time signal processing system and method, and more particularly, to a real-time signal processing system and method based on multi-channel independent component analysis.

### BACKGROUND OF THE INVENTION

There are activities continuously going on in the nerve cells of the human brains, and these cell activities emit electromagnetic waves, which are known as the brainwaves.

Electroencephalogram (EEG) is a diagram that records changes in the potential difference between two points on the skull over time, usually measured in microvolts. The generation of the potential difference is related to the potential of the cell membrane. There is a potential difference between two sides of any cell membranes. This is because the attraction between excess intracellular negative ions and extracellular positive ions results in a potential between the outer and inner layers of the cell membrane. The potential difference recorded by EEG is a collective result of millions of nerve cells near the surface of the cerebral cortex, i.e., the sum of the potentials of multiple brain cells in a specific point in time rather than the potential change of a single brain cell.

An EEG scan can record the potential changes in the brain cells in order to determine whether the brain has discharge or potential abnormalities, and this can be used by physicians to diagnose epilepsy, central nervous system and dementia, for example. However, brainwaves are easily subjected to noise interference, such as those caused by eye rolling or moving, blinking, muscle vibration, noise from the power supply and so on. Such noise will affect the processing and computing of brain-wave related applications. Therefore, eliminating noise interference is crucial to the technology of brainwave measurement.

However, the hardware currently used for eliminating noise in brainwave measurement technology tends to be cumbersome and/or costly. If software is used for reducing the cost of hardware, noise may not be removed effectively.

Therefore, it is a challenge in the art to reduce the hardware cost of the EEG detection equipment while achieving real-time processing.

### SUMMARY OF THE INVENTION

A real-time signal processing system based on multi-channel independent component analysis (ICA) includes a one-pass recursive ICA processor for performing multi-channel ICA on a set of first data to generate a plurality of second data and third data; a noise removing module coupled with the one-pass recursive ICA processor for receiving the plurality of second data, identifying noise in the plurality of second data and removing the identified noise to generate a plurality of fourth data; and a reconstruction module coupled with the noise removing module and the one-pass recursive ICA processor for receiving the plurality of fourth data and third data, and reconstructing the set of first data based on the plurality of fourth data and third data to generate a plurality of fifth data.

The set of first data are raw data, the second data are a result of the raw data after ICA with the noise, the third data are an unmixing weight matrix for separating the set of first data to generate the plurality of the second data, the fourth data are a result of the raw data after ICA and noise removal, and the fifth data are a result of the raw data after multi-channel ICA, noise removal and signal reconstruction.

In addition, the real-time signal processing system based on multi-channel ICA further includes a receiving circuit and an output circuit, wherein the receiving circuit is coupled to the one-pass recursive ICA processor to input an input signal to the one-pass recursive ICA processor and sample the input signal to obtain the set of first data, and the output circuit is coupled to the reconstruction module to receive the plurality of fifth data and output an output signal.

Moreover, the real-time signal processing system based on multi-channel ICA further includes a computation module coupled to the one-pass recursive ICA processor, the noise removing module, and the reconstruction module, wherein the one-pass recursive ICA processor uses the computation module to perform multi-channel recursive ICA in a single pass on the set of first data to generate the plurality of second data and third data, the noise removing module uses the computation module to perform noise identification on the plurality of second data and remove the identified noise to generate the plurality of fourth data, and the reconstruction module uses the computation module to perform reconstruction on the set of first data based on the plurality of fourth data and third data to generate the plurality of fifth data.

Furthermore, the receiving circuit, the one-pass recursive ICA processor, the noise removing module, the reconstruction module, the output circuit and the computation module in the real-time signal processing system based on multi-channel ICA are implemented on a single chip.

A real-time signal processing method based on multi-channel independent component analysis (ICA) includes the steps of: (1) performing multi-channel ICA on a set of first data to generate a plurality of second data and third data; (2) identifying noise in the plurality of second data and removing the identified noise to generate a plurality of fourth data; and (3) reconstructing the set of first data based on the plurality of fourth data and third data to generate a plurality of fifth data.

Before step (1) above, the method further includes receiving an input signal and sampling the input signal to obtain the set of first data, and performing the next sampling after step (1) is finished.

Compared to the prior art, the real-time signal processing system and method based on multi-channel one-pass recursive independent component analysis (ICA) can reduce hardware cost while processing the input signals effectively in real time to provide precise output signals.

### BRIEF DESCRIPTION OF THE DRAWINGS

The present invention can be more fully understood by reading the following detailed description of the preferred embodiments, with reference made to the accompanying drawings, wherein:

FIG. 1 is a schematic block diagram illustrating the basic components of a real-time signal processing system based on multi-channel one-pass recursive independent component analysis (ICA) in accordance with the present invention;

FIG. 2 is a schematic block diagram illustrating the basic components in the real-time signal processing system based on multi-channel one-pass recursive ICA in accordance with an embodiment of the present invention;

FIG. 3 is a schematic block diagram illustrating the flow of a real-time signal processing method based on multi-channel one-pass recursive ICA in accordance with an embodiment of the present invention;

FIG. 4 is a flowchart illustrating the real-time signal processing method based on multi-channel one-pass recursive ICA in accordance with the present invention;

FIG. 5 is a schematic diagram illustrating input signals of the real-time signal processing system based on multi-channel one-pass recursive ICA in accordance with the present invention; and

FIG. 6 are results of the real-time signal processing system performing the real-time signal processing method based on multi-channel one-pass recursive ICA in accordance with the present invention.

### DETAILED DESCRIPTION OF THE EMBODIMENTS

The present invention is described in the context of the following specific embodiments. Those with ordinary skills in the arts can readily understand other advantages and effects of the present invention upon reading the disclosure of this specification. The present invention can be implemented or applied in other different embodiments.

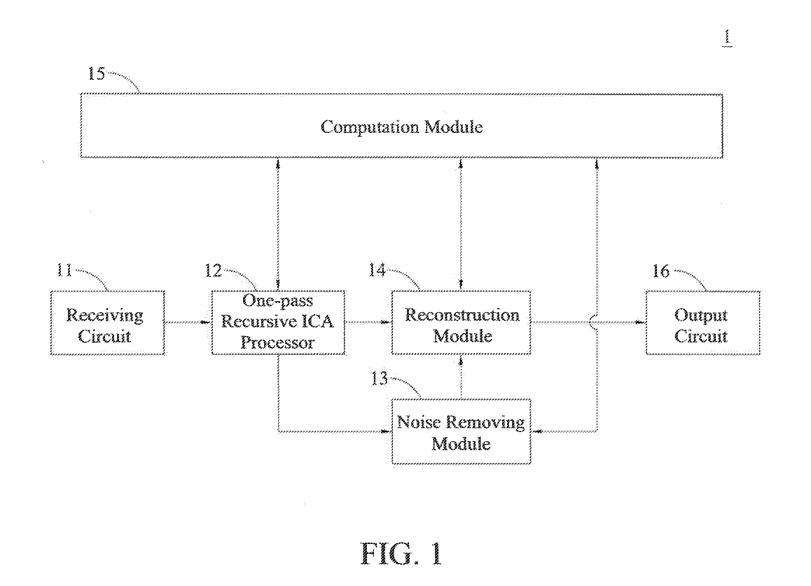

Referring to FIG. 1, a real-time signal processing system based on multi-channel one-pass recursive independent component analysis (ICA) 1 in accordance with the present invention is shown, which mainly includes a one-pass recursive ICA processor 12, a noise removing module 13, and a reconstruction module 14.

The one-pass recursive ICA processor 12 performs multi-channel recursive independent component analysis (ICA) in a single pass on a set of first data to generate a plurality of second data and third data, wherein the set of first data are raw data, the second data are the result of raw data after ICA with noise, and the third data are an unmixing weight matrix for separating the set of first data to generate the plurality of the second data.

The noise removing module 13 is coupled to the one-pass recursive ICA processor 12 to receive the plurality of second data, identify the noise in the plurality of second data and remove the identified noise to generate a plurality of fourth data, wherein the fourth data are the result of raw data after ICA and noise removal.

The reconstruction module 14 is coupled to the noise removing module 13 and the one-pass recursive ICA processor 12 to receive the plurality of fourth data and the third data, and reconstruct the first data based on the plurality of fourth data and the third data to generate a plurality of fifth data, wherein the fifth data are the result of raw data after ICA, noise removal, and reconstruction.

In addition, as shown in FIG. 1, the real-time signal processing system based on one-pass recursive ICA 1 may usually further include a receiving circuit 11, an output circuit 16, and a computation module 15.

The receiving circuit 11 is coupled to the one-pass recursive ICA processor 12 to input an input signal to the one-pass recursive ICA processor 12 and sample the input signal to obtain the set of first data. Moreover, the receiving circuit 11 waits for the one-pass recursive ICA processor 12 to finish ICA and generate the second data or third data before performing the next sampling on the input signal. The noise removing module 13 immediately performs identification and noise removal upon receiving the second data and sends to the reconstruction nodule 14. The reconstruction module 14 performs reconstruction of the set of first data based on the fourth data and the third data in real time to generate the plurality of fifth data.

The output circuit 16 is coupled to the reconstruction module 14 to receive the plurality of fifth data and output an output signal for the subsequent use.

The computation module 15 is coupled to the one-pass recursive ICA processor 12, the noise removing module 13, and the reconstruction module 14, such that the one-pass recursive ICA processor 12 uses the computation module 15 to perform the recursive ICA in a single pass on the set of first data to generate the plurality of second data and third data; the noise removing module 13 uses the computation module 15 to perform noise identification on the plurality of second data and remove the identified noise to generate the plurality of fourth data; and the reconstruction module 14 uses the computation module 15 to reconstruct the set of first data based on the plurality of fourth data and third data to generate the plurality of fifth data. Therefore, the one-pass recursive ICA processor 12, the noise removing module 13, and the reconstruction module 14 share the same computation module 15.

In FIG. 1, the receiving circuit 11, the one-pass recursive ICA processor 12, the noise removing module 13, the reconstruction module 14, the computation module 15, and the output circuit 16 are all implemented on the same chip to achieve a system on chip (SOC). Moreover, the one-pass recursive ICA processor 12, the noise removing module 13 and, the reconstruction module 14 all share the same computation module 15 to achieve low hardware cost.

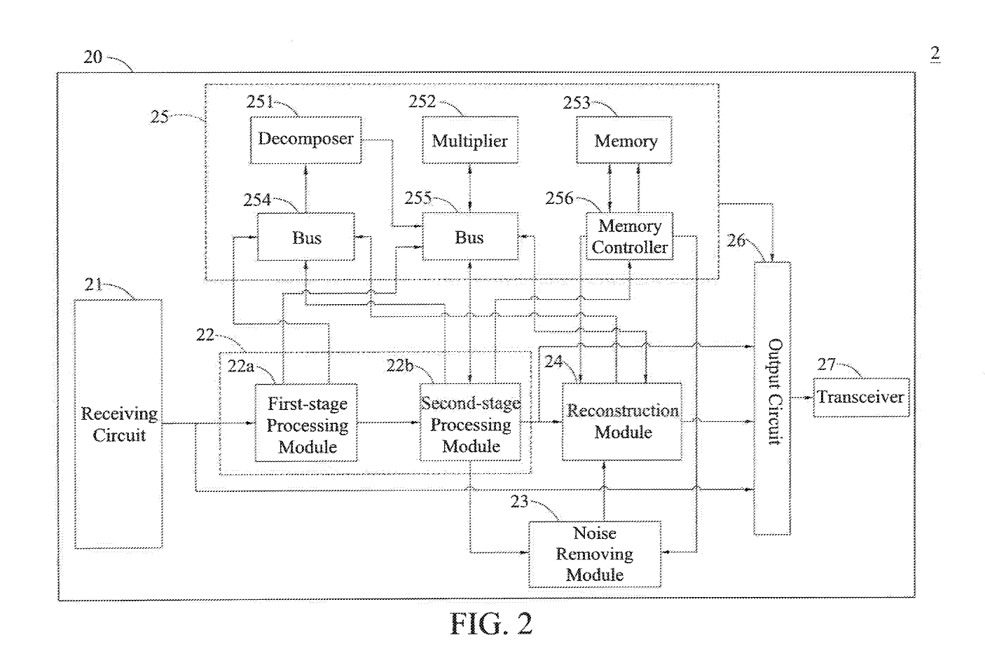

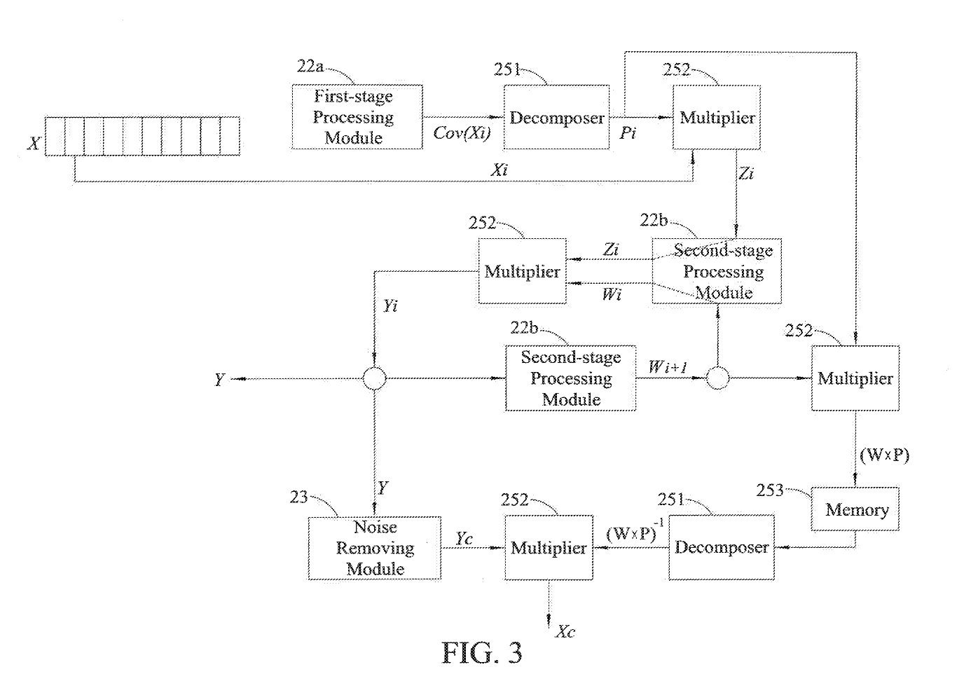

Next, FIGS. 2 and 3 further describe the real-time signal processing system based on one-pass recursive ICA in accordance with the present invention and its operational flows. FIG. 2 is a schematic block diagram illustrating the real-time signal processing system based on one-pass recursive ICA in accordance with an embodiment of the present invention, and FIG. 3 is a flowchart illustrating a real-time signal processing method based on one-pass recursive ICA in accordance with an embodiment of the present invention.

As shown in FIG. 2, a real-time signal processing system based on one-pass recursive ICA 2 in accordance with the present invention includes a receiving circuit 21, a one-pass recursive ICA processor 22 coupled to the receiving circuit 21, a noise removing module 23 coupled to the one-pass recursive ICA processor 22, a reconstruction module 24 coupled to the one-pass recursive ICA processor 22 and the noise removing module 23, a computation module 25 coupled to the one-pass recursive ICA processor 22, the noise removing module 23, and the reconstruction module 24, an output circuit 26 coupled to the reconstruction module 24 and a transceiver 27 coupled to the output circuit 26. The real-time signal processing system based on one-pass recursive ICA 2 in accordance with the present invention can be implemented on a chip 20.

As shown in FIG. 2, the computation module 25 includes a decomposer 251, a multiplier 252, a memory 253, a bus 254 coupled to the decomposer 251, the one-pass recursive ICA processor 22 and the reconstruction module 24, a bus 255 coupled to the multiplier 252, the one-pass recursive ICA processor 22 and the reconstruction module 24, and a memory controller 256 coupled to the memory 253, the one-pass recursive ICA processor 22 and the reconstruction module 24. The one-pass recursive ICA processor 22 includes a first-stage processing module 22a and a second-stage processing module 22b. The second-stage processing module 22b comprises: a one-pass recursive unmixing weight submodule for calculating the unmixing weight matrix in a single pass; a nonlinearity submodule coupled with the one-pass recursive unmixing weight submodule for determining the nonlinearity function with hyperbolic tangent; a kurtosis submodule coupled with the nonlinearity submodule for calculating the kurtosis value of the second data; a time-varying forgetting factor submodule coupled with the one-pass recursive unmixing weight submodule for calculating the forgetting factor; and a normalization submodule coupled with the one-pass recursive unmixing weight submodule for calculating the normalization of the third data. The nonlinearity submodule comprises: a mirrored nonlinearity look-up unit for determining the hyperbolic tangent of the second data; a state selected multiplexor coupled with the mirrored nonlinearity look-up unit and the output register for determining nonlinearity function in accordance with the system state of the one-pass recursive ICA processor; a kurtosis selected multiplexor coupled with the state selected multiplexor for determining nonlinearity function in accordance with the output result of the kurtosis submodule; and an output register coupled with the state selected multiplexor for buffering the determined nonlinearity function.

The flow chart of the real-time signal processing method based on one-pass recursive ICA in accordance with the present invention is described below in conjunction with FIG. 3. The receiving circuit 21 receives an input signal such as a brainwave signal. The receiving circuit 21 samples the input signal with a sampling frequency of 128 samples per second.

It should be noted that the first data X are represented by a matrix, and Xᵢ represents an element in the matrix, so X and Xᵢ both indicate the first data. Data below are represented in the similar way.

The one-pass recursive ICA processor 22 includes a first-stage processing module 22a and a second-stage processing module 22b.

The first-stage processing module 22a performs whitening process on the first data Xᵢ to generate a covariance matrix Cov(Xᵢ) of the first data Xᵢ. Then the decomposer 251 is used to perform processing on the covariance matrix Cov(Xᵢ) to generate a whitening matrix P which is equal to the inverse-square-root covariance matrix Cov(Xᵢ). Then the multiplier 252 is used to perform float-point operation on the whitening matrix Pᵢ and the first data Xᵢ to remove dependency between the data, thereby generating sixth data Z₁. The processes performed by the first-stage processing module 22a are shown in equations (1) to (3) below.

Cov(X)=E└X,X^{T}┘ (1)

P=Cov(X)^{-1/2} (2)

Z=P×X (3)

The second-stage processing module 22b performs the independent component analysis training on the sixth data Zᵢ in order to generate the third data, i.e., an unmixing weight matrix Wᵢ, and the multiplier 252 is used to carry out processing on the sixth data Zᵢ and the third data Wᵢ to generate the second data wherein the processing performed by the second-stage processing module 22b on the second data Yᵢ, the third data Wᵢ and the sixth data Zᵢ is one-pass recursive ICA algorithm. The processes performed by the second-stage processing module 22b are shown in equations (4) to (11) below. The second data Y generated are the result of the raw data X after one-pass recursive ICA with noise.

$$\begin{matrix}Y = W \times Z & ( 4 ) \\ Δ W = \frac{λ}{1 - λ} [ W - \frac{y \times {f}^{T} \times W}{1 + λ ( {f}^{T} \times y - 1 )} ] & ( 5 ) \\ W = W + Δ W & ( 6 ) \\ W = \frac{W}{‖ W ‖} = \frac{W}{{〈 W , W 〉}^{1 / 2}} = W \times {〈 W , W 〉}^{- 1 / 2} & ( 7 ) \\ λ ( n ) = \{ \frac{\frac{0.995}{0.025 n + 1} , n \leq 10000}{\frac{0.995}{0.0078125 n + 251} , n > 1000} & ( 8 ) \\ kurt ( y ) = \frac{E \{ {y}^{4} \}}{{( E \{ {y}^{2} \} )}^{2}} - 3 & ( 9 ) \\ f ( y ) = - 2 tanh ( y ) , for Super \text{-} Gaussian components & ( 10 ) \\ f ( y ) = - 2 tanh ( y ) , for Sub \text{-} Gaussian components & ( 11 )\end{matrix}$$

, wherein λ(n) is the time-varying forgetting factor, kurt(y) is the kurtosis function of the second data, f(y) is the nonlinearity function, W is the one-pass recursive unmixing weight maxtix, and equation (7) is the normalization of W.

In other words, the first data X after going through the first-stage processing module 22a and the second-stage processing module 22b can be separated into data of independent multiple channels, and among these independent multiple channels, it is not yet known which contain the signal and which contain noise.

The noise removing module 23 can perform noise identification on the second data Y and remove the identified noise to generate the fourth data Y_{c}, i.e., the result of the raw data after ICA and noise removal.

The reconstruction module 24 can use the multiplier 252 and the decomposer 251 to perform processing on the square-root matrix P and the third data W, and then use the multiplier 252 to perform processing on the fourth data Y_{c}, the processed inverse-square-root of a corvance matrix W and the third data W to generate the fifth data X_{c}, i.e., the result of the raw data X after multi-channel ICA, noise removal, and signal reconstruction. The processes performed by the reconstruction module 24 are shown in equations (12) to (17) below.

$$\begin{matrix}D ( i ) = [ d ( i ) , d ( i + 1 ) , … , d ( i + m - 1 ) ] & ( 12 ) \\ d ( D ( i ) , D ( j ) ] = max [ \vert d ( i + k ) - d ( j + k ) \vert ] & ( 13 ) \\ {B}_{i}^{m} = \{ number of d [ D ( i ) , D ( i ) ] < r \} / ( N - M - 1 ) & ( 14 ) \\ {B}^{m} ( r ) = \frac{1}{N - m} {\sum }_{i = 1}^{N - m} {B}_{i}^{m} ( r ) & ( 15 ) \\ SampEn ( m , r ) = ln [ {B}^{m} ( r ) ] - ln ( {B}^{m + 1} ( r ) ] & ( 16 ) \\ {X}_{c} = {W}^{- 1} \times {Y}_{c} = {( {ED}^{- 1} E )}^{T} \times {Y}_{c} & ( 17 )\end{matrix}$$

It should be noted that the above equations (1) to (13) are exemplary means for implementing the first-stage processing module 22a, the second-stage processing module 22b and the reconstruction module 24 of the present invention, and the present invention is not limited as such.

The computation module 25 is now further described in more details. The one-pass recursive ICA processor 22, the noise removing module 23, and the reconstruction module 24 of the present invention all share the same computation module 25. In the computation module 25, the decomposer 251 performs singular value decomposition (SVD). The decomposer 251 is designed to he shared in order to reduce hardware cost. In addition, since the accuracy of the matrix multiplication used in the present invention has a great impact on the results of the subsequent system computations, so the IEEE 754 double-precision float-point arithmetic, for example, can be used to construct the multiplier 252 in the computation module 25. The multiplier 252 is constructed with only a set of float-point multiplier, so reduction of hardware cost can be achieved. Moreover, the memory 253 in the computation module 25 stores intermediate values of internal operations, that is, to store data after being processed by the second-stage processing module 22b for use by the subsequent noise removing module 23 and the reconstruction module 24, Furthermore, the computation module 25 provides function to one of the first-stage processing module 22a, the second-stage processing module 22b, the noise removing module 23, and the reconstruction module 24 at a time, that is, each of the modules has to wait for the previous module to finish before performing its operation. Such time division multiplexing ensures these modules will not contend resources.

The output circuit 26 can receive the first data X, the second data Y and the fifth data X_{c} to output a corresponding output signal. Thus, the output circuit 26 can output three types of signals, i.e., the unprocessed signal, the signal after ICA with noise, and the signal after ICA, noise removal, and signal reconstruction. In addition, the computation module 25 also outputs a control signal to the output circuit 26 to control one of said three types of signals outputted by the output circuit 26.

The transceiver 27 sends out the output signal via a network medium such as Bluetooth and the like.

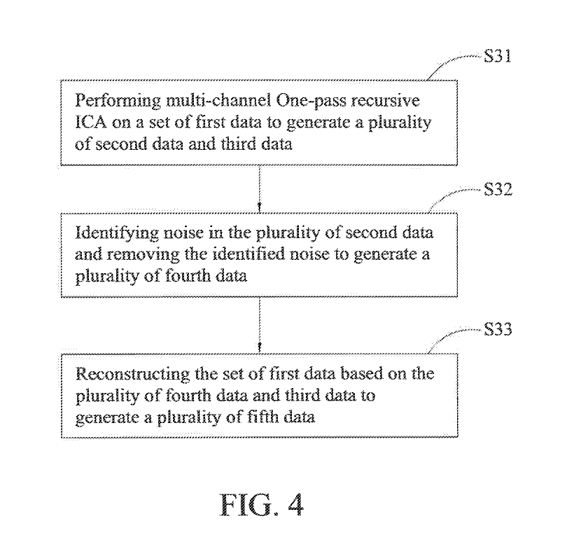

Next, the flow of the real-time signal processing method based on multi-channel one-pass recursive independent component analysis (ICA) in accordance with the present invention is generally described in FIG. 4.

In step S31, ICA is performed on a set of first data (i.e., raw data) Co generate a plurality of second data (i.e., the result of the raw data after ICA with noise) and third data (which are an unmixing weight matrix for separating the set of first data to generate the plurality of the second data). It should be noted that, before step S31, an input signal is first received and sampled to obtain the set of first data, and the next sampling is performed only after step S31 is completed. In addition, step S31 further includes that the set of first data are processed to generate a covariance matrix of the set of first data, the covariance matrix is processed to generate a whitening matrix P, which is equal to the inverse-square-root covariance matrix Cov(Xᵢ), and then a float-point operation is performed on the whitening matrix P and the set of first data to generate sixth data; and the sixth data are processed to generate the third data, and the sixth data and the third data are processed to generate the second data. Furthermore, in step S31, a one-pass recursive ICA algorithm is performed on the second data, the third data and the sixth data.

In step S32, noise identification is performed on the plurality of second data to remove the identified noise so as to generate a plurality of fourth data (i.e., the result of the raw data after ICA and noise removal).

In step S33, reconstruction is performed on the set of first data based on the plurality of fourth data and third data to generate a plurality of fifth data (i.e., the result of the raw data after ICA, noise removal and signal reconstruction). Moreover, step S33 further includes that the reconstruction module uses the multiplier and the decomposer to process an inverse matrix of the third data, and uses the multiplier to process the processed inverse matrix of the third data and the fourth data to generate the plurality of fifth data.

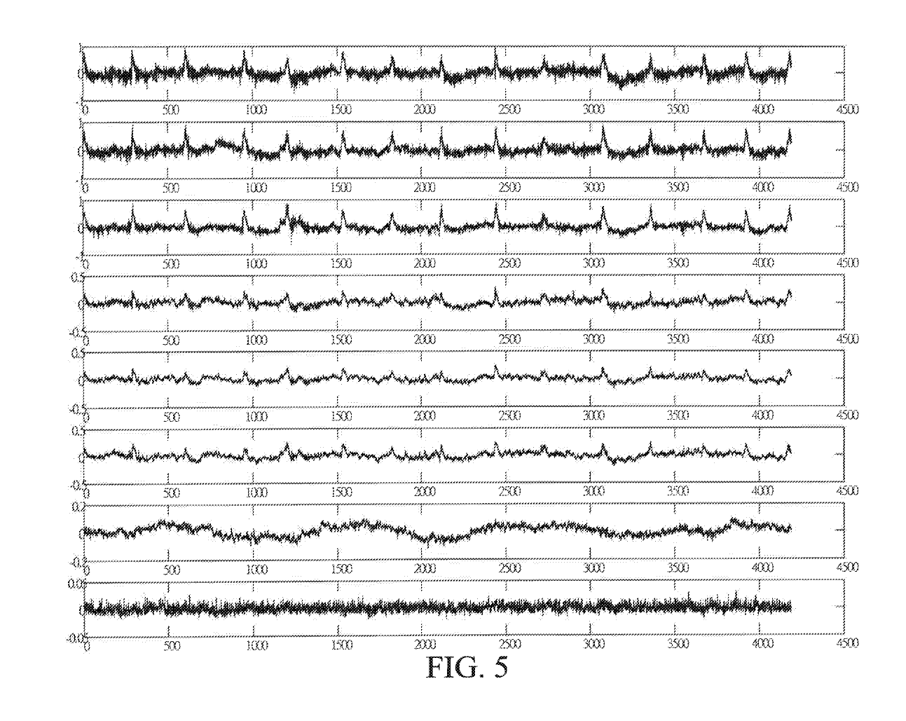

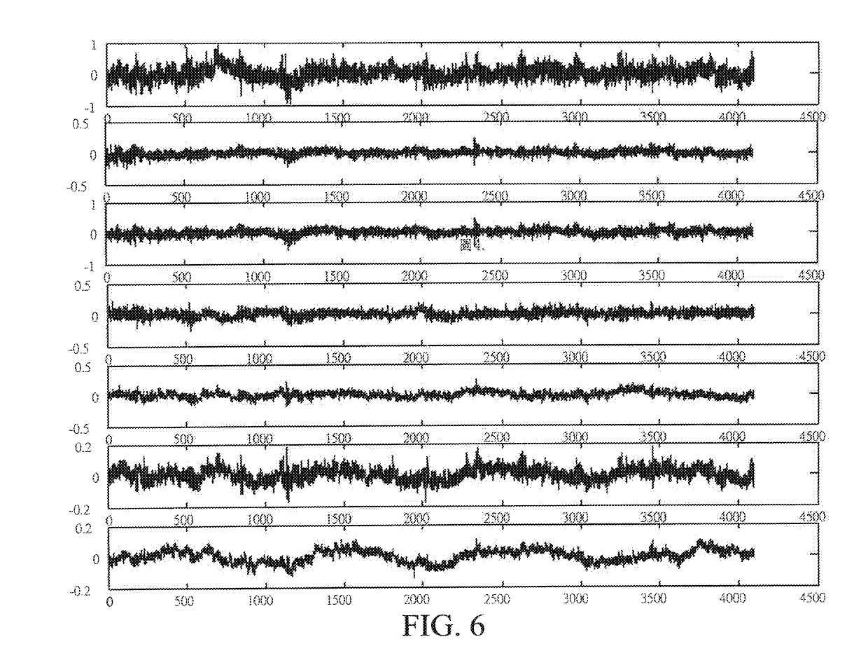

Next, the present invention is described with reference to FIGS. 5 and 6, FIG. 5 is a schematic diagram illustrating input signals of the real-time signal processing system based on multi-channel one-pass recursive independent component analysis (ICA) in accordance with the present invention, and FIG. 6 is the results of the real-time signal processing system performing the real-time signal processing method based on one-pass recursive multi-channel independent component analysis (ICA) of the present invention. In FIG. 5, brainwaves are used as examples, wherein the peaks in the signals are the noise, such as eye-movement noise. It can be seen that, after processing by the real-time signal processing system based on multi-channel one-pass recursive independent component analysis (ICA) in accordance with the present invention, the noise is removed as shown in FIG. 6 by the non-existence of peaks that were originally present in FIG. 5.

In an embodiment, the real-time signal processing system based on multi-channel one-pass recursive independent component analysis (ICA) in accordance with the present invention is designed on a chip with an area of 1829×1821 um² with an operating frequency of 50 MHz, for example. The power of the chip can be simulated with Nanosim, wherein the power is approximately 8.56 mW under 1.0 V and a working frequency of 50 MHz.

In summary, the real-time signal processing system based on multi-channel one-pass independent component analysis (ICA) in accordance with the present invention is designed on a single chip, wherein the one-pass recursive ICA processor, the modules and the circuits on the chip all share the same computation module, so that various operations can be carried out in a time division multiplexing manner. Moreover, the one-pass recursive ICA processor of the present invention performs one-pass recursive independent component analysis immediately upon receiving the first data sampled by the receiving circuit, and the receiving circuit also waits for the one-pass recursive ICA processor to finish performing the ICA on the first data and outputting the result to the noise removing module before carrying out the next data sampling. As a result, hardware resources on the chip can be efficiently used to reduce hardware cost and achieve real-time processing of signals.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A real-time signal processing system based on multi-channel one-pass recursive independent component analysis (ICA), comprising:
a one-pass recursive ICA processor for performing multi-channel recursive ICA calculation in a single pass on a set of first data to generate a plurality of second data and a plurality of third data;
a noise removing module coupled with the one-pass recursive ICA processor for receiving the plurality of the second data, identifying noise in the plurality of the second data and removing identified noise to generate a plurality of fourth data; and
a reconstruction module coupled with the noise removing module and the one-pass recursive ICA processor for receiving the fourth data and the third data, and reconstructing the set of the first data based on the fourth data and the third data to generate a plurality of fifth data.

2. The real-time signal processing system based on multi-channel one-pass recursive ICA of claim 1, wherein the set of the first data are raw data, the second data are a result of the raw data after ICA with the noise, the third data are an unmixing weight matrix for separating the set of the first data to generate the plurality of the second data, the fourth data are a result of the raw data after ICA and noise removal, and the fifth data are a result of the raw data after multi-channel ICA, noise removal and signal reconstruction.

3. The real-time signal processing system based on multi-channel one-pass recursive ICA of claim 1, further comprising a receiving circuit and an output circuit, wherein the receiving circuit is coupled to the one-pass recursive ICA processor to input an input signal to the one-pass recursive ICA processor and sample the input signal to obtain the set of the first data, and the output circuit is coupled to the reconstruction module to receive the plurality of the fifth data and output an output signal.

4. The real-time signal processing system based on multi-channel one-pass recursive ICA of claim 3, further comprising a computation module coupled to the one-pass recursive ICA processor, the noise removing module and the reconstruction module, wherein the one-pass recursive ICA processor uses the computation module to perform the multi-channel recursive ICA calculation in a single pass on the set of the first data to generate the second data and the third data, the noise removing module uses the computation module to perform noise identification on the second data and remove the identified noise to generate the fourth data, and the reconstruction module uses the computation module to perform reconstruction on the set of the first data based on the fourth data and the third data to generate the fifth data.

5. The real-time signal processing system based on multi-channel one-pass recursive ICA of claim 4, wherein the receiving circuit, the one-pass recursive ICA processor, the noise removing module, the reconstruction module, the output circuit, and the computation module are implemented on a single chip, and the one-pass recursive ICA processor includes a first-stage processing module and a second-stage processing module, and the computation module includes a decomposer, a multiplier, a memory, a memory controller, and a bus controller.

6. The real-time signal processing system based on multi-channel one-pass recursive ICA of claim 5, wherein the first-stage processing module is coupled with the receiving circuit for processing the set of the first data to generate a covariance matrix of the set of the first data, using the decomposer to process the covariance matrix to generate a square-root matrix of the covariance matrix, and using the multiplier to perform a float-point operation on the square-root matrix and the set of the first data to generate sixth data, and the second-stage processing module is coupled with the first-stage processing module so as to process the sixth data to generate the third data, and use the multiplier to process the sixth data and the third data to generate the second data.

7. The real-time signal processing system based on multi-channel one-pass recursive ICA of claim 6, wherein the second-stage processing module performs a one-pass recursive ICA algorithm on the second data, the third data and the sixth data.

8. The real-time signal processing system based on multi-channel one-pass recursive ICA of claim 6, wherein the second-stage processing module comprises:
a one-pass recursive unmixing weight submodule for calculating an unmixing weight matrix in a single pass;
a nonlinearity submodule coupled with the one-pass recursive unmixing weight submodule for determining a nonlinearity function with a hyperbolic tangent;
a kurtosis submodule coupled with the nonlinearity submodule for calculating a kurtosis value of the second data;
a time-varying forgetting factor submodule coupled with the one-pass recursive unmixing weight submodule for calculating a forgetting factor; and
a normalization submodule coupled with the one-pass recursive unmixing weight submodule for calculating the normalization of the third data.

9. The real-time signal processing system based on multi-channel one-pass recursive ICA of claim 8, wherein the nonlinearity submodule comprises:
a mirrored nonlinearity look-up unit for determining a hyperbolic tangent of the second data;
a state selected multiplexor coupled with the mirrored nonlinearity look-up unit and an output register for determining a nonlinearity function in accordance with a system state of the one-pass recursive ICA processor; and
a kurtosis selected multiplexor coupled with the state selected multiplexor fur determining a nonlinearity function in accordance with an output result of the kurtosis submodule,
wherein the output register is coupled with the state selected multiplexor for buffering the determined nonlinearity function.

10. The real-time signal processing system based on multi-channel one-pass recursive ICA of claim 5, wherein the reconstruction module uses the multiplier and the decomposer to process an inverse matrix of the third data, and uses the multiplier to process the processed inverse matrix of the third data and the fourth data to generate the plurality of the fifth data.

11. A real-time signal processing method based on multi-channel one-pass recursive independent component analysis (ICA), comprising the steps of:
(1) performing multi-channel one-pass recursive ICA on a set of first data to generate a plurality of second data and a plurality of third data;
(2) identifying noise in the second data and removing the identified noise to generate a plurality of fourth data; and
(3) reconstructing the set of the first data based on the fourth data and the third data to generate a plurality of fifth data.

12. The real-time signal processing method based on multi-channel one-pass recursive ICA of claim 11, before step (1), further comprising receiving an input signal and sampling the input signal to obtain the set of the first data, and performing a next sampling after step (1) is finished, and wherein step (1) further comprises:
(1-1) processing the set of the first data to generate a covariance matrix of the set of the first data, and processing the covariance matrix to generate a square-root matrix of the covariance matrix, and performing a float-point operation on the square-root matrix and the set of the first data to generate sixth data; and
(1-2) processing the sixth data to generate the third data and processing the sixth data and the third data to generate the second data.
